(19) United States
(12) Patent Application Publication
(10) Pub. No.: US 2022/0339413 A1
(43) Pub. Date: Oct. 27, 2022

(54) DRUG COATED BALLOON

(71) Applicant: W. L. Gore & Associates, Inc., Newark, DE (US)

(72) Inventors: Steven M. Alston, Jr., Flagstaff, AZ (US); Theresa A. Holland, Flagstaff, AZ (US); Peter D. Traylor, Flagstaff, AZ (US)

(21) Appl. No.: 17/764,655

(22) PCT Filed: Nov. 11, 2020
(86) PCT No.: PCT/US2020/059965

§ 371 Date: Mar. 29, 2022

Related U.S. Application Data
(60) Provisional application No. 62/934,294, filed on Nov. 12, 2019.

(51) Int. Cl. A61M 25/10; A61L 29/04; A61L 29/16; A61K 31/337

(52) U.S. Cl. CPC A61M 25/1029; A61L 29/049; A61L 29/16; A61K 31/337; A61M 2025/105; A61M 2025/1031; A61M 2205/0238; A61L 2300/216; A61L 2300/416; A61L 2300/802; A61L 2420/02

(57) ABSTRACT

The present disclosure is directed toward drug coated balloons, and in particular to drug coated balloons having a drug coating layer that primarily uses therapeutic agents alone for improving the quality of treatments in which drug coated balloons are utilized. Particular aspects may be directed to drug coated balloon having an outer surface, and a drug coating layer on the outer surface of the balloon. The drug coating layer includes at least one therapeutic agent and is substantially free of excipients.

### CROSS-REFERENCE TO RELATED APPLICATION

This application is a national phase application of PCT Application No. PCT/US2020/059965, internationally filed on Nov. 11, 2020, which claims the benefit of Provisional Application No. 62/934,294, filed Nov. 12, 2019, which are incorporated herein by reference in their entireties for all purposes.

### FIELD

The present disclosure relates generally to drug coated balloons, and more specifically to drug coated balloon systems wherein the drug coating does not include excipients.

### BACKGROUND

Vascular diseases, such as atherosclerosis, artery occlusion, and restenosis, are a leading cause of human mortality and morbidity. Vascular diseases arise from a variety of causes, and in some cases, necessitate surgical or endovascular intervention. Trauma to the vascular system can also necessitate surgical intervention to treat the traumatized anatomy. The treatment of vascular disease at a local, rather than systemic, level is often preferred. Systemic administration of drugs can produce unwanted side effects, when compared to the local administration of a drug to a target tissue to treat vascular disease. The utilization of a drug-coated endovascular medical device has become a standard technique in the treatment of vascular disease. In particular, a common treatment for vascular disease is the short-term or long-term contact of a tissue with an endovascular medical device, such as a balloon or a stent, respectively, that is coated with a drug that prevents or reduces vascular disease at the site of contact. The short-term contact of vascular medical devices including catheter-based balloons, are often undertaken to treat vascular diseases and vascular trauma, and the long-term contact, e.g., implantation, of endovascular medical devices including vascular grafts, stent-grafts, and stents. Upon contact of the endovascular medical device with a diseased vascular tissue, the drug elutes from the endovascular medical device into the surrounding tissue at the site of contact, thereby treating the vascular disease at a local, rather than systemic, level.

Drug coated balloons (DCBs) are an example of a drug-coated endovascular medical device. The literature discloses the use of DCBs for the treatment of vascular diseases, including coronary artery disease and peripheral artery disease (see e.g., U.S. Pat. No. 5,102,402 to Dror et al.). Dror et al. discloses placing a DCB in a blood vessel lumen to treat the vessel wall, inflating the balloon, and contacting the balloon surface with the luminal vessel wall to deliver a drug into the blood vessel wall. The dosing of the drug to the treatment site using DCBs can be highly variable and unpredictable immediately after implantation or deployment, and local drug levels in the vascular tissue can be highly variable and unpredictable over an extended time. It is therefore desirable to have improved DCBs and techniques for treating vascular disease that are reliable and reproducible in drug dosing.

In order to improve upon conventional DCBs and techniques for treating vascular disease, DCBs may be constructed of one or more layers of material selected to provide certain properties to optimize performance of the DCB in some particular way, depending on the application. In the case of multi-layer or composite balloons, the multiple layers within the composite may be different materials to obtain a blend of physical and/or chemical properties to optimize performance. U.S. Patent Application Publication No. 2016/0106961 to Cully et al. discloses composite or layered DCBs. The described composite materials can comprise a porous layer adhered to a blow moldable polymer, such as a composite material that comprises an expanded fluoropolymer layer that is adhered to a blow moldable polymer through a stretch blow molding process. Additionally, U.S. Patent Application Publication No. 2014/0271775 to Cleek et al. discloses composite or layered DCBs comprising substrates having oriented drug crystals of high aspect ratio habit. The described composite materials can comprise a substrate comprising a porous microstructure and an amount of crystalline paclitaxel (PTX) comprising hollow crystal habits associated with the substrate.

Although the multi-layer or composite balloons described in Cully et al. and Cleek et al. achieve some success in the reliability and reproducibility of drug dosing, many of the conventional multi-layer or composite balloons demonstrate lower than optimal drug application and retention. This phenomenon arises due to a number of factors including a non-uniform coating of the drug on the substrate, particulation or loss of the drug coating during deployment of the DCB, and/or a non-uniform or incomplete transfer of the drug coating to the surface of the vessel lumen, which ultimately results in lower than optimal drug application and retention. Accordingly, the need exists for improved DCBs and techniques for increasing vascular permeability for drug application and retention.

The drug coatings of conventional DCBs include excipients or other additives to facilitate drug retention on the balloon surface before use, drug release from the balloon surface during use, and drug transport into the tissue.

### SUMMARY

It has been thought that excipients are necessary for a drug formulation on drug coated balloons for retention of the drug on the balloon surface before use, to facilitate the release of the drug formulation from the balloon to the treated tissue during use, and for retention of the drug in the treated tissue. The effect of excipients on paclitaxel release from drug coated balloons, tissue retention, drug coating loss during procedure, and late lumen loss were investigated using various methods further detailed herein. Testing included comparisons of composite balloons including ePTFE and commercially available balloons. Different drug formulations utilizing different amounts of excipients were also compared. Testing revealed that the presence of excipients had no significant effect on any of the parameters tested.

According to an example (“Example 1”) of the present disclosure, a drug coated balloon is disclosed. The drug coated balloon comprises a balloon having an outer surface comprised of expanded polytetrafluoroethylene and a drug coating layer on the outer surface of the balloon. The drug coating layer comprises at least one therapeutic agent and is substantially free of excipients.

The balloon of the drug coated balloon of Example 1 may be comprised of a composite of expanded polytetrafluoroethylene and nylon. The at least one therapeutic agent may comprise paclitaxel. Where the at least one therapeutic agent comprises paclitaxel, the balloon may be configured to deliver the paclitaxel to a tissue to reduce a cellular proliferative response associated with restenosis. Where the at least one therapeutic agent comprises paclitaxel, the drug coating layer may further comprise a therapeutic agent selected from docetaxel, protexel, arsenic trioxide, thalidomide, atorvastatin, cerivastatin, Fluvastatin, betamethasone diproprionate, dexamethasone 21-palmitate, sirolimus, everolimus, zotarolimus, biolimus, or temisirolimus.

The drug coating layer of the drug coated balloon of Example 1 may contain from 0% to 4.75% by weight of the excipients. The drug coating layer of the drug coated balloon of Example 1 may contain from 0% to 4.75% by weight of excipients, wherein the excipients are selected from fatty acids and their derivatives and urea. The outer surface of the balloon of the drug coated balloon of Example 1 may further comprise nylon. The outer surface of the balloon of the drug coated balloon may comprise expanded polytetrafluoroethylene. The drug coating layer may penetrate the outer surface of the balloon by an average penetration depth of 2 μm to 10 μm. About 80% of the drug coating layer may release from the balloon in about 100 minutes following implantation

The drug coating layer may comprise microcrystals in a haystack orientation having a random and a substantial absence of uniformity in placement on the outer surface of the balloon. Where the drug coating layer comprises microcrystals, a majority of the microcrystals may each have a major dimension length that is at least 10 times greater than a major dimension width. The major dimension length of the majority of the microcrystals may be at least 13 times or at least 15 times greater than the major dimension length. The major dimension width of the majority of the microcrystals may be between 0.5 μm and 2 μm. The microcrystals may have a random and substantial absence of uniformity in angles from the outer surface, and a majority of the microcrystals may project from the outer surface at an angle of 50 degrees to 15 degrees.

In another example (“Example 2”) of the present disclosure, a drug coated balloon is disclosed. The drug coated balloon comprises a balloon having an outer surface and a drug coating layer on the outer surface of the balloon. The drug coated layer comprises at least one therapeutic agent and is substantially free of excipients. The drug coating layer further comprises microcrystals in a haystack orientation having a random and a substantial absence of uniformity in placement on the outer surface of the balloon.

The outer surface of the balloon of the drug coated balloon of Example 2 may comprise nylon. The outer surface of the balloon may comprise expanded polytetrafluoroethylene. The majority of the microcrystals of the drug coating layer of the drug coated balloon of Example 2 may each have a major dimension length that is at least 10 times greater than a major dimension width. The major dimension length of the majority of the microcrystals may be at least 13 times or at least 15 times greater than the major dimension length. The major dimension width of the majority of the microcrystals may be between 0.5 μm and 2 μm. The microcrystals may have a random and substantial absence of uniformity in angles from the outer surface, and a majority of the microcrystals project from the outer surface at an angle of 50 degrees to 15 degrees.

In yet another example (“Example 3”) of the present disclosure, a method for preparing a vessel for drug application is disclosed. The method comprises the steps of solubilizing at least one therapeutic agent in a solvent to produce a solution; coating an outer surface of a medical balloon with the solution; and evaporating the solvent, leaving a drug coating layer comprising the at least one therapeutic agent on the outer surface of the balloon so that the drug coating layer comprises microcrystals in a haystack orientation having a random and substantial absence of uniformity in placement on the outer surface of the balloon. The solution is substantially free of excipients.

The solvent of the method of Example 3 may comprise acetone. Where the solvent comprises acetone, the solvent may further comprise water. Where the solvent comprises both acetone and water, the solvent may comprise approximately 75% acetone and approximately 25% water. Where the solvent comprises both acetone and water, the solvent may further comprise dioxane. Where the solvent comprises acetone, dioxane, and water, the solvent may comprise approximately 58% acetone, 14% dioxane, and 28% water. The balloon of Example 3 may be comprised of a composite material comprising a fluoropolymer and nylon. The at least one therapeutic agent of Example 3 may comprise paclitaxel.

In another example (“Example 4”) of the present disclosure, a drug coated balloon is disclosed. The drug coated balloon comprises a balloon having an outer surface comprised of expanded polytetrafluoroethylene and a drug coating layer on the outer surface of the balloon. The drug coating layer comprises at least one therapeutic agent and from 0% to 4.75% by weight of fatty acids and their derivatives. The fatty acids and their derivatives of Example 4 may be selected from monocarboxylic acids, polysorbates, and shellac.

In yet another example (“Example 5”) of the present disclosure, a drug coated balloon is disclosed. The drug coated balloon comprises a balloon having an outer surface comprised of expanded polytetrafluoroethylene and a drug coating layer on the outer surface of the balloon. The drug coating layer comprises at least one therapeutic agent and from 0% to 4.75% by weight of urea.

The foregoing Examples are just that and should not be read to limit or otherwise narrow the scope of any of the inventive concepts otherwise provided in the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows the assembly of a microtubule by polymerization in accordance with some aspects of the present disclosure;

FIG. 2A shows a DCB in accordance with some aspects of the present disclosure;

FIG. 2B shows a cross-section of composite material forming the DCB embodiment shown in FIG. 2A;

FIG. 2C shows a cross-section of the DCB embodiment shown in FIG. 2A;

FIG. 2D shows a thickness characterization of a composite material forming a DCB in accordance with some aspects of the present disclosure;

FIGS. 3A and 3B show microcrystalline morphology on non-porous and porous balloon substrates in accordance with some aspects of the present disclosure.

FIG. 4 shows a balloon catheter assembly in accordance with some aspects of the present disclosure;

FIG. 5A is a graph comparing tissue retention of a drug delivered by different DCBs tested in Comparative Example A in accordance with some aspects of the present disclosure;

FIG. 5B is a table comparing tissue retention of a drug delivered by different DCBs tested in Comparative Example A in accordance with some aspects of the present disclosure;

FIG. 5C is a graph comparing tissue retention of a drug delivered by different DCBs tested in Comparative Example A in accordance with some aspects of the present disclosure;

FIG. 6 is a table comparing drug release of different DCBs tested in Comparative Example A in accordance with some aspects of the present disclosure;

FIG. 7 is a graph comparing different DCBs tested in Comparative Example A in accordance with some aspects of the present disclosure;

FIG. 8 is a table of target loading percentages of different DCBs tested in Example B in accordance with some aspects of the present disclosure;

FIG. 9 is a graph of the cumulative in vitro PTX release from different DCBs tested in Example B in accordance with some aspects of the present disclosure;

FIG. 10 is a graph of the PTX remaining on different DCBs tested in Example B after the said DCBs were inflated in vivo in accordance with some aspects of the present disclosure;

FIG. 11 is a graph of the PTX remaining in treated tissue one day after treatment of the tissue by different DCBs tested in Example B in accordance with some aspects of the present disclosure;

FIG. 12A is a graph of the PTX concentration in treated tissue over varying time periods after treatment of the tissue by different DCBs tested in Example C in accordance with some aspects of the present disclosure;

FIG. 12B is a graph of the total amount of PTX remaining in an artery over varying time periods after treatment of tissue within the artery by different DCBs tested in Example C in accordance with some aspects of the present disclosure;

FIG. 13 is a graph of the cumulative in vitro PTX release of different DCBs tested in Example D in accordance with some aspects of the present disclosure

FIG. 14A is a graph of the PTX remaining on different DCBs tested in Example D in accordance with some aspects of the present disclosure, after passing through an introducer valve;

FIG. 14B is a graph of the PTX remaining on different packaging sheaths of corresponding, different DCBs tested in Example D in accordance with some aspects of the present disclosure, after the DCBs pass through an introducer valve;

FIG. 14C is a graph of the PTX remaining on different introducer valves of corresponding, different DCBs tested in Example D in accordance with some aspects of the present disclosure, after the DCBs each pass through an introducer valve; and

FIG. 15 is a graph of the late lumen loss of treated arteries treated by different DCBs tested in Example E in accordance with some aspects of the present disclosure.

### DETAILED DESCRIPTION

### Definitions and Terminology

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior/exterior direction.

The term “majorly” or “majority” indicates at least half or 50%.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, any of the present devices, systems, and methods that “comprises,” “has,” “includes” or “contains” one or more elements possesses those one or more elements but is not limited to possessing only those one or more elements. Likewise, an element of a device, system, or method that “comprises,” “has,” “includes” or “contains” one or more features possesses those one or more elements but is not limited to possessing only those one or more elements. Likewise, an element of a device, system, or method that “comprises,” “has,” “includes” or “contains” one or more features possesses those one or more features but is not limited to possessing only those one or more features.

Any of the present devices, systems, and methods can consist of or consist essentially of rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

Furthermore, a structure that is capable of performing a function or that is configured in a certain way is capable or configured in at least that way but may also be capable or configured in ways that are not listed.

As used herein, “nominal diameter” means the approximate diameter of the balloon at the nominal inflation pressure. Beyond this state, pressure increases (e.g., up to the rated burst pressure) result in less than a 20% increase in diameter, less than a 15% increase in diameter, or less than a 10% increase in diameter. Typically, the nominal diameter is the labeled diameter as indicated on the instructions for the end user, e.g., a clinician.

A “microcrystal haystack” or the phrase “microcrystals in a haystack orientation” is defined as a plurality of microcrystals, the majority of which are oriented at less than a 20° angle from a surface on which they are positioned. In addition, the majority of the plurality of microcrystals each have a major (overall) dimension length that is at least five (5) times greater than the major (overall) dimension width of the microcrystal.

The terms “angle,” “projection angle,” “angle of projection,” and the like, are the geometric angle that a projecting object has relative to the outermost plane of a surface.

“Placement” and the like means a rotation or offset that an object has relative to a central axis of the outermost plane of a surface.

A “uniform distribution,” “uniformly distributed,” and the like, means that a percentage by volume of the microcrystals present within a given volume on a surface of a substrate is maintained across the surface within a certain percentage, where the certain percentage includes 3, 10, and/or 20 percent. In one particular example, microcrystals may be “uniformly distributed” across the substrate if each given volume on the surface of the substrate contains 65% to 68% by volume of microcrystals (i.e., within 3%), 65% to 75% by volume of microcrystals (i.e., within 10%), and/or 65% to 85% by volume of microcrystals (i.e., within 20%).

An “excipient” is any inactive ingredient in a drug coating. An excipient is a substance formulated alongside a therapeutic agent to stabilize, bulk up, or confer a therapeutic enhancement on the therapeutic agent, such as facilitating drug absorption, reducing viscosity, or enhancing solubility. Such excipients include saturated or unsaturated fatty acids and their derivatives, such as monocarboxylic acid salts derived from a monocarboxylic acid (e.g., stearic acid) and a base (e.g., tris(hydroxymethyl)aminomethane) (“tris”)), polyoxyethylene sorbitan fatty acid esters (polysorbates) derived from sorbitol and a fatty acid (e.g., lauric, stearic, and oleic acids), and shellac (e.g., aleuritic, jalaric and shellolic acids). Another excipient includes urea, for example.

The section headers in the description below are not meant to be read in a limiting sense, nor are they meant to segregate the collective disclosure presented below. The disclosure should be read as a whole. The headings are simply provided to assist with review, and do not imply that discussion outside of a particular heading is inapplicable to the portion of the disclosure falling under that header. This disclosure is not meant to be read in a restrictive manner. For example, the terminology used in the application should be read broadly in the context of the meaning those in the field would attribute such terminology.

### DESCRIPTION OF VARIOUS EMBODIMENTS

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### I. INTRODUCTION

In various embodiments, a balloon is provided for that comprises an outer surface and a drug coating layer on the outer surface of the balloon. Conventional approaches for the treatment of vascular disease include the utilization of a drug coated balloon (DCB). However, a problem associated with traditional DCBs is the difficulty in maintaining tissue retention of the drug and reducing drug loss in the biological fluid, e.g., blood, during DCB transport to a therapeutic location, particularly when factors are present that increase rates at which drugs associate with and/or dissociate from receptors (i.e., reversible binding and binding kinetics). For example, the reversible binding of drugs to receptors within tissue is a function of dose and residence time.

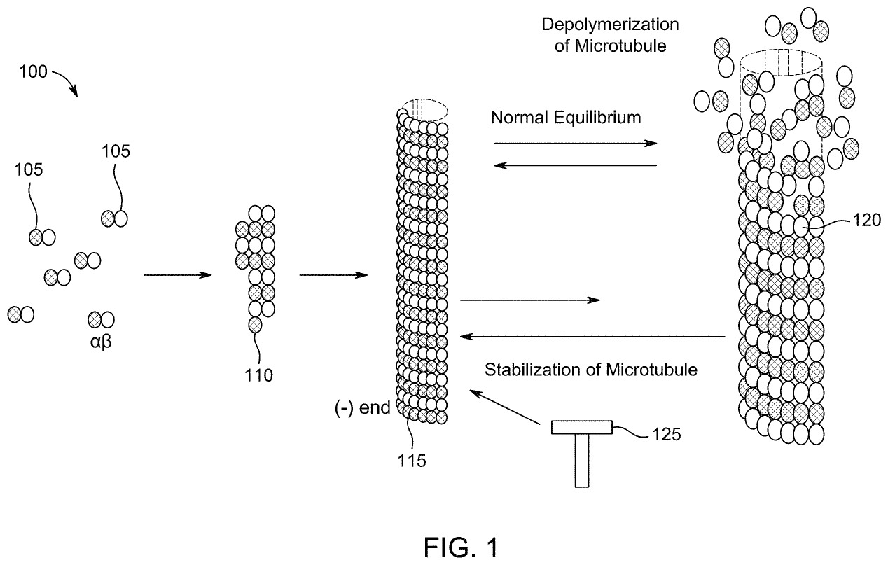

FIG. 1 shows how the reversible binding of a drug such as a taxane to receptor sites within a microtubule may affect tissue retention. The assembly 100 of a microtubule by the polymerization of αβ tubulin heterodimers 105 occurs in two phases: nucleation 110 and elongation 115. Formation of a short polymerization nucleus precedes elongation or polymer growth at each end by the reversible, noncovalent addition of tubulin subunits. For net polymer elongation, the association of tubulin heterodimers 105 into the growing microtubule is faster than microtubule depolymerization. However, at a steady state, growth of microtubule polymer due to αβ-heterodimer addition is counterbalanced by shrinkage due to disassembly into αβ-tubulin subunits. Thus, a polymerized microtubule 120 switches between episodes of growth and shrinkage, a property called dynamic instability (i.e., a normal equilibrium for the microtubule).

Taxanes 125 such as paclitaxel (PTX) are microtubule-binding drugs that target specific sites within the lumen of polymerized microtubules 120. The taxanes 125 act by binding to GDP-bound β-tubulin molecules and stabilizing them by changing their conformation to a more stable GTP-bound β-tubulin structure. The taxanes cytotoxic effect is attributed to their ability to bind tubulin, stabilize the protofilaments leading to microtubule over-polymerization, and ultimately death by apoptosis. However, without being bound by theory, the reversible binding of taxanes 125 to the lumen of the polymerized microtubules 120 may hinder diffusion of the taxanes 125 along the microtubule axis, and thus affect tissue retention of the taxanes 125.

In order to address the aforementioned tissue retention and drug loss problems, U.S. Publication No. 2017/0367705 discloses a coating morphology comprising microcrystals in a haystack orientation that maintains the drug on a surface of the DCB during transport, inflation, and deflation of the DCB to a therapeutic location, e.g., an arterial wall, while also increasing tissue retention of the drug transferred from the DCB. This coating morphology may reduce or eliminate the need for a removable cover on the DCB and/or a porous layer over a surface of the DCB, which are techniques that have been used to traditionally maintain the drug on the surface of the DCB during transport, inflation, and deflation of the DCB.

Without being bound by theory, it has been demonstrated that the microcrystalline surface coating morphology comprising microcrystals in a haystack orientation may determine distribution of the drug on the inner surface of the blood vessel and in some manner may maximize tissue retention of the drug. In addition, the microcrystalline surface coating morphology comprising microcrystals in a haystack orientation may minimize drug loss during transport and/or during inflation and deflation without the need of an additional cover/layer over the drug coating, as demonstrated in the below Comparative Example A. Thus, it has been demonstrated that utilization of DCBs having microcrystalline surface coating morphology comprising microcrystals in a haystack orientation on a fluoropolymer surface minimized drug loss during transport, inflation, and deflation without the need of an additional removable cover/layer over the drug coating. These results demonstrate that a DCB with a fluoropolymer surface having a porous microstructure may promote microcrystalline growth from the porous microstructure and/or overcome particulation of the drug coating during transport, inflation, and deflation.

Moreover, it has been demonstrated that the microcrystalline surface coating morphology comprising microcrystals in a haystack orientation may improve distribution of the drug on the inner surface of the blood vessel when positioned on either a porous or a non-porous polymer layer of a balloon.

Furthermore, it has been demonstrated that tissue retention from DCB benefits from a microcrystalline surface coating morphology comprising microcrystals in a haystack orientation but not due to increased dose exposure, which has been hypothesized. Although one skilled in the art would expect that a DCB that delivers a higher initial dose exposure over that of another DCB will also have a higher retainable dose at 1 hour over that of the other DCB, the results of DCBs with a fluoropolymer surface and a microcrystalline surface coating morphology comprising microcrystals in a haystack orientation provided a synergistic increase in tissue retention, as shown in the below Comparative Example A. This demonstrates that microcrystalline surface coating morphology on a DCB with a fluoropolymer surface having a porous microstructure may prepare the artery for enhanced absorption and/or overcome restrictions of poor drug deliverance.

In addition to the tissue retention problem described above, it has been previously thought that excipients were required within the formulation of the drug coating layer to enhance long-term stabilization, to bulk up solid formulations that contain potent active ingredients (thus often referred to as “bulking agents,” “fillers,” or “diluents”), or to confer a therapeutic enhancement on the therapeutic agent in the final dosage form, such as facilitating drug absorption, reducing viscosity, or enhancing solubility.

Without being bound by theory, it has been demonstrated that the presence of excipients on a balloon with a fluoropolymer surface, especially as part of a microcrystalline surface coating morphology comprising microcrystals in a haystack orientation, has little effect on therapeutic agent retention in a treated tissue. Further, it has been demonstrated that a balloon with a fluoropolymer surface and a drug coating without excipients may be passed through an introducer valve without increased drug loss relative to devices having a drug coating with excipients. Moreover, it surprisingly has now been discovered that excipients may have minimal, if any, effect on in vivo and in vitro therapeutic drug release from the balloon. Thus, the drug coating layer of the present disclosure may be substantially free of excipients.

### II. DRUG COATED BALLOONS (DCBS)

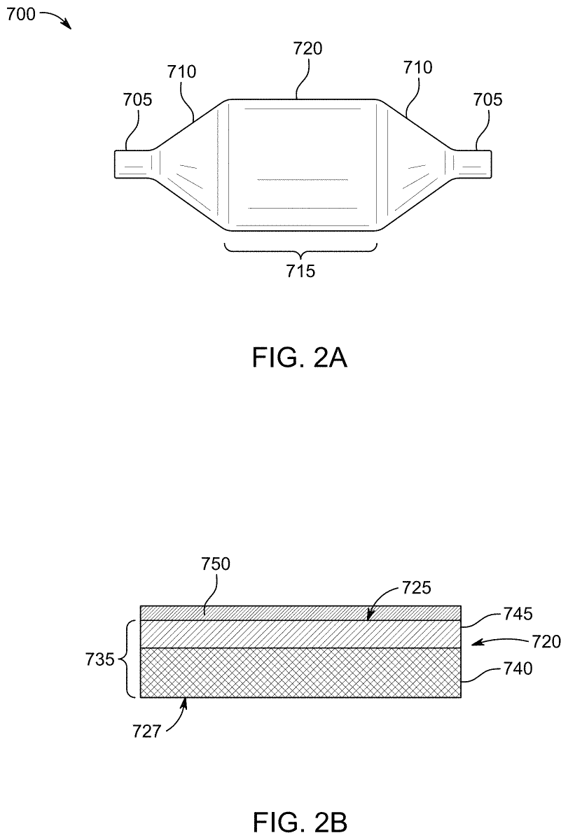

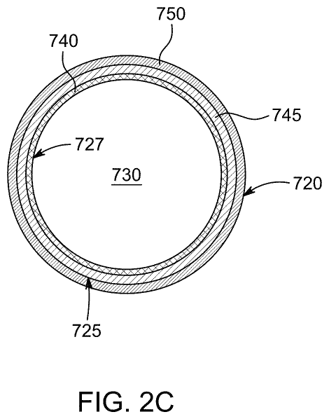

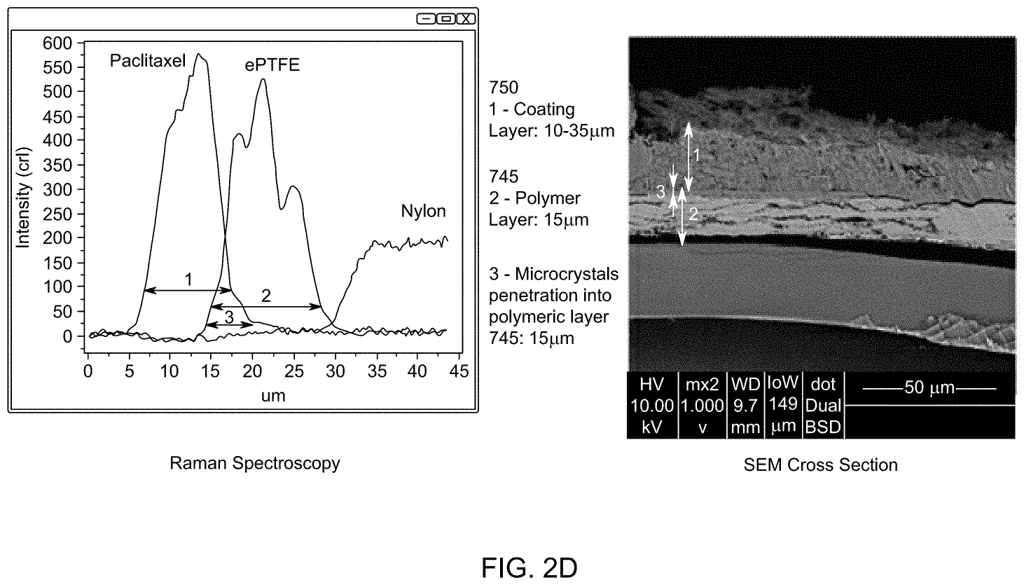

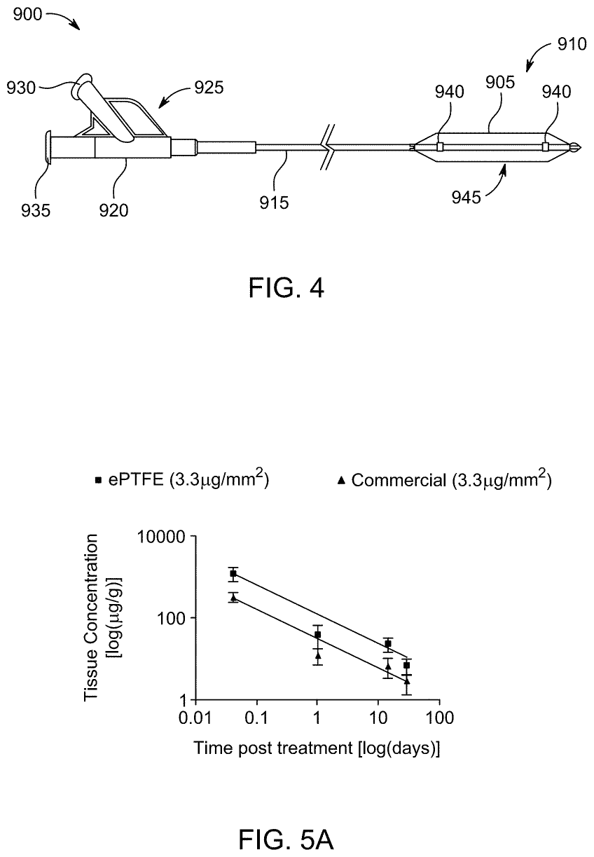

In various embodiments, a medical device is provided that comprises a balloon having an outer surface and a drug coating layer on the outer surface of the balloon. The drug coating may comprise microcrystals in a haystack orientation applied even across the outer surface of the balloon. The balloon can have any appropriate dimension and size for the clinical application. In some embodiments, the balloon is substantially cylindrical along the working length. In some embodiments, the balloon may be a wrapped balloon, for example a balloon helically wrapped at an angle between 20 degrees and 90 degrees or 40 degrees and 70 degrees, or other suitable wrap angles. In some embodiments, the balloon may be a wrapped balloon that is wrapped at approximately a 90-degree angle (i.e., a cigarette wrapped balloon). As shown in FIG. 2A, the illustrative balloon 700 has two opposed leg portions 705 that are integrally connected to shoulder/tapered portions 710. For the purposes of this disclosure, “working length” is defined as the length of the straight body section 715 of the balloon 700 which comprises the approximate length between the opposed shoulder/tapered portions 710. Leg portions 705, shoulder/tapered portions 710, and straight body section 715 define an “overall length” of the balloon 700. The working length of the balloon 700 can be about 10 mm to about 150 mm or more. Similarly, a nominal diameter of the balloon 700 can be about 2 mm to about 30 mm or more. By way of example, the balloon 700 can have a 4 mm diameter and a 30 mm working length, or alternatively, an 8 mm diameter and about a 60 mm working length. Of course, the balloon 700 of the present disclosure can be constructed at any dimensions appropriate for the specific use. The balloon 700 may be attached or mounted to a catheter 915 (as shown in FIG. 4) at the leg portions for delivery of a drug coating via inflation of the balloon 700 in the vasculature. The catheter 915 may have one or more lumens, one of which may be in communication with the chamber of the balloon 700 for supplying inflation fluid to inflate the balloon 700.

With reference to FIGS. 2A, 2B, 2C, and 2D, the balloon 700 may further comprise a balloon wall 720 comprising an outer surface 725 and an inner surface 727. The balloon wall 720 defines a chamber 730 and may be constructed of a layered material 735. In some embodiments, the layered material 735 comprises a thermoplastic polymeric layer 740 at least partially adhered to a substrate or polymeric layer 745 in an overlying relationship to each other. In certain embodiments, the layered material 735 can be created through a stretch blow molding process, as described in U.S. Patent Application Publication No. 2016/0106961. In other embodiments, the layered material 735 can be created by wrapping (e.g., a helical wrap) one layer around another layer, for example, the polymeric layer 745 may be wrapped around the thermoplastic polymeric layer 740, as described in U.S. Patent Application Publication No. 2016/0143759 A1. The layered material 735 may be constructed by blow mold or wrapping such that a thickness of the thermoplastic polymeric layer 740 is from 10 μm to 40 μm, for example, from 15 μm to 35 μm or about 30 μm, and a thickness of the polymeric layer 745 is from 5 μm to 50 μm, for example, from 10 μm to 30 μm or about 15 μm.

In some embodiments, the thermoplastic polymeric layer 740 defines the inner surface 727 of the balloon wall 720, which serves as a bladder to retain the inflation fluid, and thus is composed of an impermeable or fluid-tight material. In accordance with such aspects, the polymeric layer 745 or other layer of material (e.g., a second polymeric layer) defines the outer surface 725 of the balloon wall 720. In other embodiments, the polymeric layer 745 defines the inner surface of the balloon wall 720, which serves as a bladder to retain the inflation fluid, and thus is composed of an impermeable or fluid-tight material. In accordance with such aspects, the thermoplastic polymeric layer 740 or other layer of material (e.g., a second polymeric layer) may define the outer surface 725 of the balloon wall 720.

In various embodiments, a coating layer 750 (e.g., a drug coating) is distributed evenly across at least a portion of the outer surface 725 of the layered material 735. For example, as shown in FIGS. 2B, 2C, and 2D, a coating layer 750 may be distributed evenly across at least a portion of the outer surface 725 of the polymeric layer 745. In some embodiments, the coating layer 750 is distributed evenly across only substantially the working length of the balloon 700. In other embodiments, the coating layer 750 is distributed evenly across substantially the working length of the balloon 700 and at least a portion of the leg portions 705 and/or shoulder/tapered portions 710. An “even distribution,” “distributed evenly,” and the like, means that a thickness of the coating layer 750 across the portion of the outer surface 725 is maintained within a certain percentage of a specified thickness, where the percentage includes 3, 10, and 20 percent. In certain embodiments, a thickness of the coating layer 750 is from 5 μm to 50 μm, for example, from 10 μm to 35 μm.

As shown in FIG. 2D, the thickness of the layered material 735 and the coating layer 750 was studied by Raman spectroscopy and scanning electron microscopy techniques, and it was found that the coating layer 750 may have an average penetration depth from 2 μm to 10 μm (e.g., about 5 μm) of the polymeric layer 745 having a porous microstructure, and thus may infiltrate the outermost layer of a porous microstructure cover (e.g., expanded polytetrafluoroethylene (ePTFE)). In contrast, the coating layer 750 does not penetrate into a polymeric layer 745 having a nonporous microstructure, and thus would be fully disposed on the outermost layers (e.g., outer two layers) of a non-porous microstructure cover (e.g., Nylon). Accordingly, without being bound by theory, a polymeric layer 745 having a porous microstructure, such as ePTFE, provides a sponge-like scaffold for the coating layer 750 (e.g., PTX) with a coating penetration to a depth of about 5 μm.

In some embodiments, a thickness of the coating layer 750 and the polymeric layer 745 is from 5 μm to 45 μm, for example, from 10 μm to 35 μm or about 25 μm. In some embodiments, a thickness of the coating layer 750, the thermoplastic polymeric layer 740, and the polymeric layer 745 is from 30 μm to 60 μm, for example, from 40 μm to 55 μm or about 45 μm. A ratio of a thickness of the thermoplastic polymeric layer 740 to a thickness of the polymeric layer 745 may be from 1.5:1 to 2.5:1, for example about 2:1 or about 30 μm of thermoplastic polymeric layer 740 to 15 μm of polymeric layer 745. A ratio of a thickness of the thermoplastic polymeric layer 740 to a thickness of (the polymeric layer 745 and the coating layer 750) may be from 1:1 to 1.7:1, for example about 1.2:1 or about 30 μm of thermoplastic polymeric layer 740 to 25 μm of (the polymeric layer 745 and the coating layer 750).

The thermoplastic polymeric layer 740 may be comprised of a compliant, semi-compliant, or non-compliant thermoplastic polymer. Suitable thermoplastic polymers include polymers that are medical grade and are blow moldable. Examples of suitable thermoplastic polymers can include polymethyl methacrylate (PMMA or Acrylic), polystyrene (PS), acrylonitrile butadiene styrene (ABS), polyvinyl chloride (PVC), modified polyethylene terephthalate glycol (PETG), cellulose acetate butyrate (CAB); semi-crystalline commodity plastics that include polyethylene (PE), high density polyethylene *HDPE), low density polyethylene *LDPE or LLDPE), polypropylene (PP), polymethylpentene (PMP); polycarbonate (PC), polyphenylene oxide (PPO), modified polyphenylene oxide (Mod PPO), polyphenylene ether (PPE), modified polyphenylene ether (Mod PPE), thermoplastic polyurethane (TPU); polyoxymethylene (POM or Acetal), polyethylene terephthalate (PET, Thermoplastic Polyester), polybutylene terephthalate (PBT, Thermoplastic Polyester), polyimide (PI, Imidized Plastic), polyamide-imide (PAI, Imidized Plastic), polybenzimidazole (PBI, Imidized Plastic); polysulfone (PSU), polyetherimide (PEI), polyether sulfone (PES), polyaryl sulfone (PAS); polyphenylene sulfide (PPS), polyether ether ketone (PEEK); fluoropolymers that include fluorinated ethylene propylene (FEP), ethylene chlorotrifluoroethylene (ECTFE) ethylene tetrafluoroethylene (ETFE), polychlorotrifluoroethylene (PCTFE), polyvinylidene fluoride (PVDF), perfluoroalkoxy (PFA), or combinations, copolymers, or derivatives thereof. Other commonly known medical grade materials include elastomeric organosilicon polymers, and polyether block amide (e.g., PEBAX®). In particular, polyamides may include Nylon 12, Nylon 11, Nylon 9, Nylon 6/9, and Nylon 6/6. In certain embodiments, PET, Nylon, and PE may be selected for medical balloons used in coronary angioplasty or other high pressure applications. The specific choice of materials may depend on the desired characteristics/intended application of the balloon.

The polymeric layer 745 may be comprised of a compliant, semi-compliant, or non-compliant polymer. Suitable polymers include a porous microstructure or a non-porous microstructure. In embodiments having a porous microstructure (referred to herein as a “porous layer”), suitable polymers include fluoropolymers, including, without limitation, perfluoroelastomers and the like, polytetrafluoroethylene (PTFE) and the like, as well as expanded fluoropolymers, including ePTFE. In embodiments having a non-porous microstructure (referred to herein as a “non-porous layer”), suitable polymers include polyamides, including, without limitation, Nylon 12, Nylon 11, Nylon 9, Nylon 6/9, and Nylon 6/6.

The architecture of the porous microstructure may be selected based on the needs of the intended application. In some embodiments, the porous microstructure may be substantially fibrillated (e.g., a non-woven web having a microstructure of substantially only fibrils, some fused at crossover points or with smaller nodal dimensions). In other embodiments, the porous microstructure can comprise large nodes or large densified regions that may have an impact on the extent of compressibility/collapsibility of the material during blow molding. In still other embodiments, the porous microstructure can be a node and fibril microstructure somewhere between the aforementioned embodiments. In some embodiments, the porous microstructure can have an “open” microstructure such that the outer polymeric layer 745 can have more loft and/or a drug coating layer 750 can have more void space to occupy near the surface of the outer polymeric layer 745. Other examples of porous architectures can be fibrous structures (such as woven or braided fabrics), non-woven mats of fibers, microfibers, or nanofibers, flash spun films, electrospun films, and other porous films.

In some embodiments, the porous microstructure may be comprised of expanded fluoropolymers or expanded polyethylene (see e.g., U.S. Pat. No. 6,743,388 to Sridharan et al.). Non-limiting examples of expanded fluoropolymers include, but are not limited to, ePTFE, expanded modified PTFE, and expanded copolymers of PTFE. Patents have been filed on expandable blends of PTFE, expandable modified PTFE, and expanded copolymers of PTFE, such as, for example, U.S. Pat. No. 5,708,044 to Branca; U.S. Pat. No. 6,541,589 to Baillie; U.S. Pat. No. 7,531,611 to Sabol et al.; U.S. Pat. No. 8,637,144 to Ford; and U.S. Pat. No. 8,937,105 to Xu et al.

The polymeric layer 745 may be formed from a tubular member of a polymer having the porous or nonporous microstructure. The tubular member can be formed as an extruded tube or can be film-wrapped. The tubular member can have circumferential, helical, or axial orientations of the microstructure. In various embodiments, the tubular member can be formed by wrapping a film or tape and the orientation can be controlled by the angle of the wrapping. The tubular member can be circumferentially wrapped or helically wrapped. When a porous material is wrapped helically versus circumferentially or axially, the degree of compliancy in a given direction can be varied and can influence the overall compliancy of the composite. (As used herein, the term “axial” is interchangeable with the term “longitudinal.” As used herein, “circumferential” means an angle that is substantially perpendicular to the longitudinal axis.)

The coating layer 750 may be comprised of at least one natural, semi-synthetic, or synthetic therapeutic agent (e.g., at least one drug). The functional characteristic of the coating layer 750 is to allow for release of at least one therapeutic agent to the tissue of a vascular wall during balloon inflation (e.g., treatment via percutaneous transluminal angioplasty in patients with obstructive disease of the peripheral arteries). In certain embodiments, the therapeutic agent is either lipophilic (partition coefficient between n-butanol and water >10) or displays very poor water solubility (<10 mg/ml, 20° C.). The wording “at least one therapeutic agent (or therapeutic agent preparation)” means that a single therapeutic agent or mixtures of different therapeutic agents are included. Thus, various therapeutic agents may be applied or combined if different pharmacological actions are required or efficacy or tolerance is to be improved.

Therapeutic agents suitable for use in the coating layer 750 may include inhibitors of restenosis or cell proliferation (e.g., an anti-mitotic drug or anti-proliferative drug) such as vinco alkaloids, e.g., colchicine, podophyllotoxin, griseofulvin, antimitotic alkaloid agents, and antimicrotubule alkaloid agents, and taxanes, e.g., PTX, docetaxel, and protaxel. In certain embodiments, the therapeutic agent comprises PTX or arsenic trioxide. Alternatively, therapeutic agents suitable for use in the coating layer 750 may include specific inhibitors of neovascularization such as thalidomide, statins like atorvastatin, cerivastatin, fluvastatin, or anti-inflammatory drugs like corticoids or even lipophilic derivatives of corticoids such as betamethasone diproprionate or dexamethasone 21-palmitate, and Limus drugs, especially immune-suppressants and mitosis inhibitors like mTOR inhibitors such as sirolimus, everolimus, zotarolimus, biolimus, and temsirolimus. That is, for some embodiments, the therapeutic agent comprises PTX, a taxane, docetaxel, vinca alkaloids, colchicine, podophyllotoxin, griseofulvin, antimitotic alkaloid agents, antimicrotubule alkaloid agents, protaxel, arsenic trioxide, thalidomide, atorvastatin, cerivastatin, Fluvastatin, betamethasone diproprionate, dexamethasone 21-palmitate, sirolimus, everolimus, zotarolimus, biolimus, or temsirolimus. As should be understood, the at least one therapeutic agent may include structural analogs, related substances, degradants, and derivatives of any of the afore-mentioned drugs.

The drug coating layer 750 may be substantially free of excipients, which may allow for a thinner drug coating layer 750 and delivery of a lower total coating mass. For example, the drug coating layer 750 may be substantially free of ingredients intended to enhance long-term stabilization, to bulk up solid formulations that contain potent active ingredients, or to confer a therapeutic enhancement on the therapeutic agent in the final dosage form, such as facilitating drug absorption, reducing viscosity, or enhancing solubility. Such excipients include saturated or unsaturated fatty acids and their derivatives, such as monocarboxylic acid salts derived from a monocarboxylic acid (e.g., stearic acid) and a base (e.g., tris), polyoxyethylene sorbitan fatty acid esters (polysorbates) derived from sorbitol and a fatty acid (e.g., lauric, stearic, and oleic acids), and shellac (e.g., aleuritic, jalaric and shellolic acids). Another excipient includes urea, for example. Thus, the drug coating layer 750 may be substantially free of fatty acids and their derivatives (including monocarboxylic acids, polysorbates, and shellac), urea, and/or other excipients. Stated differently, the drug coating layer 750 may consist of the at least one therapeutic agent without any excipients (i.e., 0% by weight of excipients in the drug coating layer 750) or consist essentially of the at least one therapeutic agent with insignificant amounts of excipients (i.e., 4.75% by weight or less of excipients in the drug coating layer 750). Thus, in certain embodiments, the drug coating layer 750 may contain from 0% to 4.75% by weight of excipients, such as about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, 4.5% by weight, or 4.75% by weight.

In various embodiments, the formulation for the coating layer 750 comprises at least one therapeutic agent and a volatile solvent. The choice of the solvent may be useful for the crystal morphology of the coating layer 750 in a dry state and adherence and release of the therapeutic agent from the surface of the medical device. Accordingly, the solvent may include acetone, dioxane, tetrahydrofuran (THF), water, and mixtures thereof.

Accordingly, in some embodiments, the solvent comprises acetone, dioxane, THF, and water. In other embodiments, the solvent consists essentially of acetone, dioxane, THF, and water. In other embodiments, the solvent comprises acetone, dioxane, and water. In other embodiments, the solvent consists essentially of acetone, dioxane, and water. In other embodiments, the solvent comprises acetone and water. In yet other embodiments, the solvent consists essentially of acetone and water. In some embodiments, the solvent comprises from about 35% to about 85% by volume of acetone, for example, about 75% or about 0.75 v/v, and from about 5% to about 35% by volume of water, for example, about 25% or about 0.25 v/v. In other embodiments, the solvent comprises from about 35% to about 65% by volume of acetone, for example, about 58% or about 0.58 v/v, from about 5% to about 35% by volume of water, for example, about 28% or about 0.27 v/v, and from about 5% to about 30% by volume of dioxane, for example, about 14% or about 0.14 v/v. However, one skilled in the art will understand that modifications of this formulation may be acceptable provided that the modifications do not change the primary constituents and may include, without limitation, modifications that involve similar derivatives and analogs to these primary constituents. In order for the drug coating layer 750 to be substantially free of excipients, the formulation used to form the drug coating layer 750 may contain only the at least one therapeutic agent and the solvent while being substantially free of excipients.

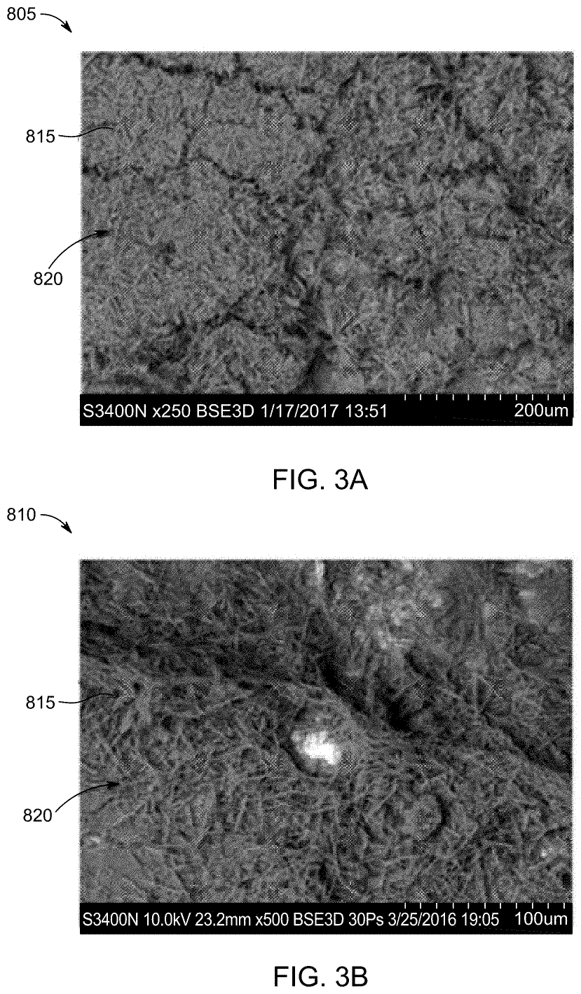

As discussed herein, various embodiments are directed to DCBs advantageously having a coating morphology that maintains the drug on a surface of the DCB during transport, inflation, and deflation of the DCB to a therapeutic location, e.g., an arterial wall, while also increasing tissue retention of the drug transferred from the DCB. As shown in FIGS. 3A and 3B, the coating layer formulation, established in accordance with the various embodiments described herein, provides a substantially similar microcrystalline morphology on non-porous substrates (e.g., Nylon) 805 (FIG. 3A) and porous balloon substrates (e.g. ePTFE) 810 (FIG. 3B). The microcrystalline morphology may comprise microcrystals 815 in a haystack orientation 820. For example, the microcrystals 815 may be uniformly distributed across the substrate 805, 810 and demonstrate a random and a substantial absence of uniformity (non-uniform) in placement on the substrate 805, 810 and/or random and a substantial absence of uniformity (non-uniform) in angle of projection from the substrate 805, 810. In some embodiments, a percentage by volume of the microcrystals 815 present within a given volume on the surface of the substrate 805, 810 is from about 50% to about 100%, for example, from about 65% to about 85%.

A majority of the microcrystals 815 may extend from a surface of the substrate 805, 810 at an angle of less than 20° (thus the crystals lay relatively flat on the substrate 805, 810). In other embodiments, a majority of the microcrystals 815 extend from the surface of the substrate 805, 810 at an angle of 50° to 17°, 50° to 15°, less than 15°, less than 10°, or less than 8°. Additionally, a majority of the microcrystals 815 each have a major dimension length that is at least five (5) times greater than the major dimension width of the microcrystal 815. In other embodiments, a majority of the microcrystals 815 each have a major dimension length that is at least ten (10) times greater than the major dimension width of the microcrystal 815. In other embodiments, a majority of the microcrystals 815 each have a major dimension length that is at least 13 or at least 15 times a major dimension width. Additionally, a majority of the microcrystals 815 optionally each have a major dimension length that is between 12 μm and 22 μm, for example between 14 μm and 20 μm or about 17 μm, and a majority of the microcrystals 815 each have a major dimension width that is between 0.5 μm and 2.0 μm, for example between 0.8 μm and 1.6 μm or about 1.3 μm.

Returning to FIG. 2D, the coating layer 750, and thus microcrystals (e.g., microcrystals 815), may penetrate into the outer 2-7 μm (e.g., about 5 μm) of a polymeric layer 745 having a porous microstructure similar to porous substrate 810 of FIG. 3B, and thus may infiltrate the outermost layers (e.g., outer two layers) of a porous microstructure cover (e.g., ePTFE). In contrast, the coating layer 750, and thus microcrystals 815, do not penetrate into a polymeric layer 745 having a non-porous microstructure similar to non-porous substrate 805 of FIG. 3A, and thus would be disposed substantially on the outermost layers (e.g., outer two layers) of a non-porous microstructure cover (e.g., Nylon). Accordingly, a polymeric layer 745 having a porous microstructure, such as ePTFE, provides a sponge-like scaffold for the coating layer 750 (e.g., PTX) with a coating penetration to a depth of about 5 μm, which may assist in minimizing the loss of the drug during tracking and deployment as well as provide for better application.

Advantageously, the coating formulation and morphology allows for the therapeutic agent to adhere firmly enough to the substrate (e.g., porous substrate 810 and non-porous substrate 805) to tolerate mechanical stress during production including folding of balloons, packaging, shipping to customers, and during final clinical use, which involves passage through a narrow hemostatic valve, an introductory sheath or guiding catheter, and a variable distance of possibly tortuous and narrow blood vessels. Moreover, the coating morphology is economical and efficient in manufacture as it does not require added costs or manufacturing steps to provide: a roughened balloon surface to enhance adherence, protective sheaths or membranes, or other physical or chemical methods to enhance adherence of the therapeutic agent to the balloon surface.

As shown in FIG. 4, a balloon catheter assembly 900 may comprise a balloon 905, for example the balloon 700 as described with respect to FIGS. 2A, 2B, 2C, and 2D, mounted on a distal section 910 of a catheter 915. The balloon catheter assembly 900 may also include a hub assembly 920 positioned on a proximal section 925 of the catheter 915. The hub assembly 920 may include an inflation port 930 that is in fluid communication with an inflation lumen of the catheter 915. The inflation lumen of the catheter 915 may be in fluid communication with an inner region of the balloon 905 such that an inflation medium may be inserted into the inflation port 930 to inflate the balloon 905. The hub assembly 920 may also include a second port 935 that is in communication with a central lumen of the catheter 915. The central lumen of the catheter 915 may extend form the proximal section 925 of the catheter 915 to the distal section 910 of the catheter 915 and may receive a guidewire. In some aspects, the central lumen of the catheter 915 may also be used for flushing an inflation medium from the balloon 905. One or more radiopaque markers 940, for example, but not limited to radiopaque platinum-iridium markers, may be positioned on the balloon 905 to indicate a working length of the balloon 905 and facilitate fluoroscopic visualization of the balloon 905 during delivery and placement. In some embodiments, the radiopaque marker may be positioned on the catheter 915 to indicate a working length of the balloon 905. As described above with respect to FIGS. 2B, 2C, and 2D, the balloon 905 includes a coating layer 945 on at least a portion of an outer surface of the balloon 905.

### III. METHODS OF TREATMENT USING DCBS

In accordance with various embodiments, a DCB (e.g., as a part of a balloon catheter assembly) may be utilized to provide one or more treatments at a desired site in a body lumen. The DCB may include a balloon having a coating layer on an outer surface of the balloon, as described with respect to FIGS. 2A, 2B, 2C, and 2D. Techniques performing the one or more treatments may include positioning the DCB on a distal section of a balloon catheter assembly, as described with respect to FIG. 4, and advancing the DCB within a body lumen to a desired site. The location of the DCB or catheter may be monitored or tracked as it is advanced in the body lumen using radiopaque elements positioned on the DCB or catheter. Once the DCB has advanced to the desired site, the DCB may provide a single treatment (or inflation), or in other embodiments, may provide multiple or repeated treatments (or inflations) at the desired site. In various embodiments where the DCB provides a single treatment at the desired site, the DCB may be inflated for from 20 to 200 seconds, e.g., approximately 45 seconds, approximately 60 seconds, approximately 90 seconds, approximately 180 seconds, or other suitable lengths of time for each treatment (or inflation). Following the one or more treatments, the DCB may be deflated and withdrawn from the body lumen.

As should be understood, the multiple or repeat treatments may be performed by inflating a single DCB multiple times at the same treatment site (as described in the above process) or by inflating multiple DCBs at a same treatment site multiple times (repeatedly performing the above process a number of times). For example, other aspects of the disclosure are directed to techniques of using the described DCBs in a sequential medical procedure. Such techniques can comprise passing the balloon catheter device with a DCB mounted thereon through an anatomical conduit or vessel to the desired position and inflating the described DCB to a nominal diameter once or sequentially. The method can further comprise expanding the balloon and delivering, upon inflation, a therapeutic agent that is on the outer surface of the balloon to a surrounding tissue or endovascular device. The method can further comprise sequentially retracting the balloon catheter device from the anatomical conduit or vessel and passing another DCB mounted to the balloon catheter device through the anatomical conduit or vessel to the desired position and inflating the subsequent balloon to a nominal diameter once or sequentially.

### IV. TEST METHODS

It should be understood that although certain methods and equipment are described below, other methods or equipment determined suitable by one of ordinary skill in the art may be alternatively utilized.

### V. EXAMPLES

Without intending to limit the scope of the embodiments discussed herein, the systems and methods implemented in various embodiments may be better understood by referring to the following examples.

### Comparative Example A

The preclinical performance of an embodiment of a DCB was evaluated, where PTX formulated with excipients was applied to an ePTFE porous surface of a composite ePTFE/Nylon balloon.

Methods: PTX/excipient coated balloons (5 mm×40 mm and 6 mm×40 mm ePTFE— W.L. Gore, Flagstaff Ariz.) with dose densities of 3.5 μg/mm² (labeled amount) or 3.3 μg/mm² (measured amount) of PTX were inflated in peripheral arteries of Yorkshire swine. PTX/excipient coated balloons (Commercial, Medtronic IN.PACT™ Admiral™ Paclitaxel-coated PTA Balloon Catheter) with dose densities of 3.5 μg/mm² (labeled amount) or 3.3 μg/mm² (measured amount) of paclitaxel were inflated in peripheral arteries of Yorkshire swine. Arteries received a clinical dose via a single treatment of inflation for 60 seconds. Animals were euthanized from 1 hour to 30 days post-treatment and subjected to comprehensive necropsies. The balloons were collected for analysis of paclitaxel release, and treated arteries, along with other tissues, were collected for bioanalysis or processed for histologic and scanning electron microscopy (SEM) evaluation.

Additionally, PTX/excipient coated balloons (5 mm×40 mm and 6 mm×40 mm ePTFE— W.L. Gore, Flagstaff Ariz.) with dose densities of 3.5 μg/mm² were inflated and deflated at a benchtop. Particulates of the coating from each balloon were collected for analysis after the inflation/deflation.

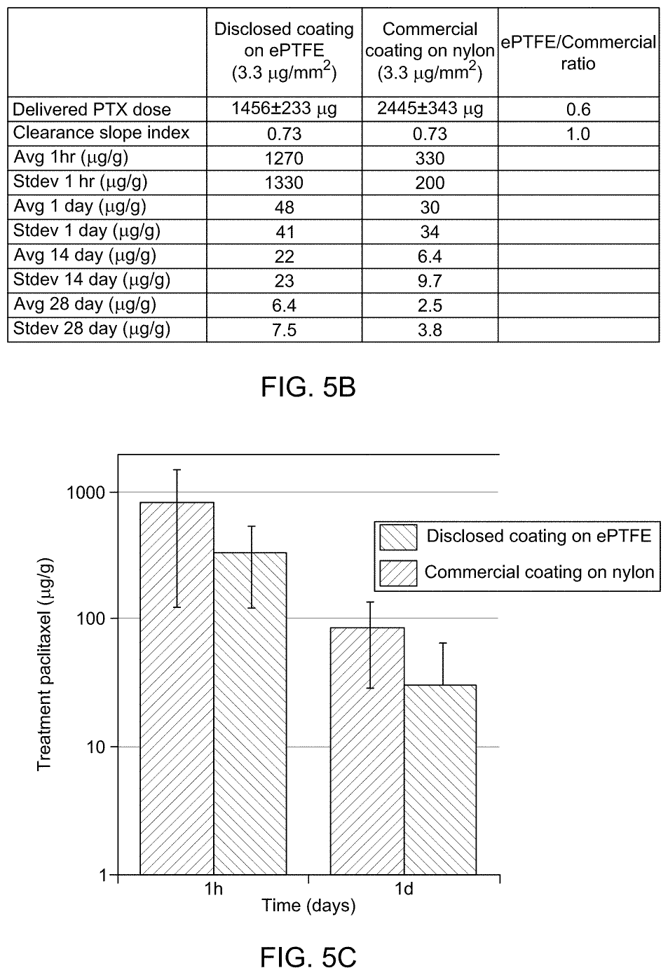

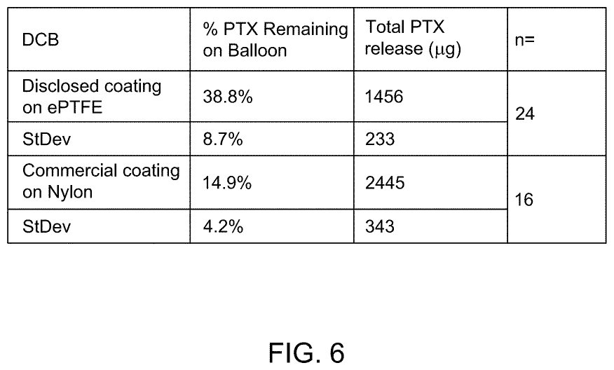

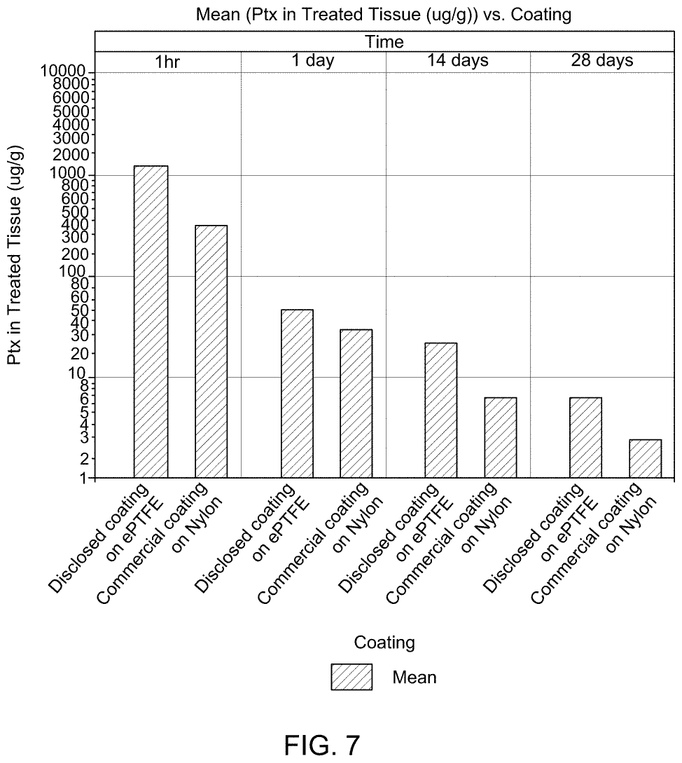

Results: As shown in FIGS. 5-7 the PTX/excipient coated balloons according to embodiments of the disclosure (ePTFE— W.L. Gore, Flagstaff Ariz.) at a dosage density of 3.5 μg/mm² released 61.2% of a loaded dose amount, for 1456 μg±233 μg of PTX and achieved a tissue concentration or dose amount of 1270 μg/g at one hour, and 48 μg/g at 1 day, 22 μg/g at 14 days, and 6.4 μg/g at 28 days respectively, and a clearance slope index of 0.73. The PTX/excipient coated balloons (Commercial) at a similar dosage density of 3.5 μg/mm², released 85.1% of a loaded dose amount, for example 2445 μg±343 μg of PTX, and achieved a tissue concentration or dose amount of 330 μg/g of PTX at 1 hour, 30 μg/g at 1 day, 6.4 μg/g at 14 days, and 2.5 μg/g at 28 days respectively, and a clearance slope index of 0.73.

While the PTX/excipient coated balloons (Commercial) delivered a greater initial PTX dose compared to that of the PTX coated balloons according to embodiments of the disclosure (ePTFE— W.L. Gore, Flagstaff Ariz.) (an ePTFE/Commercial ratio of 0.6), the PTX/excipient coated balloons (ePTFE— W.L. Gore, Flagstaff Ariz.) were able to achieve 3.8 fold greater maximum retainable dose at 1 hour compared to the PTX/excipient coated balloons (Commercial) (an ePTFE/Commercial ratio of 3.8) and a 1.6 fold greater maximum retainable dose at 1 day compared to the PTX/excipient coated balloons (Commercial) (an ePTFE/Commercial ratio of 1.6). Although one skilled in the art would expect that the PTX/excipient coated balloons (Commercial) delivering a higher initial dose of PTX over that of the PTX/excipient coated balloons (ePTFE— W.L. Gore, Flagstaff Ariz.) would also have a higher retainable dose at 1 hour or 1 day over that of the PTX/excipient coated balloons (ePTFE)— W.L. Gore, Flagstaff Ariz.), the results of DCBs with a fluoropolymer surface and a microcrystalline surface coating morphology provided a synergistic increase in tissue retention.

Surprisingly, the fluoropolymer surface and a microcrystalline surface coating morphology of the PTX/excipient coated balloons (ePTFE— W.L. Gore, Flagstaff Ariz.) produced a 3.8-fold higher retainable drug deliver at 1 hour and a 1.6-fold higher retainable drug deliver at 1 day over that of the PTX/excipient coated balloons (Commercial). Moreover, the PTX coated balloons (ePTFE— W.L. Gore, Flagstaff Ariz.) showed no signs of particulation after inflation/deflation at a benchtop, whereas the PTX/excipient coated balloons (Commercial) demonstrated particulation.

Conclusions: Overall, treatment in peripheral arteries with DCB embodiments of this disclosure at a dose density of 3.5 μg/mm², resulted in acceptable acute device performance, no adverse safety events, and pharmacokinetics similar to other PTX/excipient coated balloons. Imaging of the balloon surfaces coated with similar PTX doses (3.5 μg/mm²) revealed different morphologies. Tissue retention from embodiments of this disclosure benefits form the fluoropolymer surface and a microcrystalline surface coating morphology of the coated balloons but not due to increased dose exposure. The microcrystalline surface coating morphology in some manner may decrease particulation and increase tissue retention of the drug. The similar clearance rates may be attributable to similar tissue clearance mechanisms for both type of DCBs, which demonstrates the importance of early delivery and retention of the drug.

### Example B

The preclinical performance of an embodiment of a DCB was evaluated, where PTX was applied to an ePTFE porous surface of a composite ePTFE/Nylon balloon. Six of the testing groups contained varying amounts of stearic acid and tris excipients, while one of the testing groups did not contain excipients.

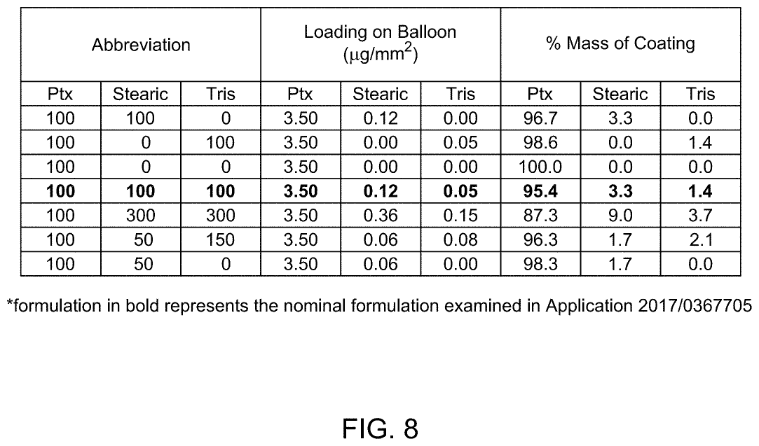

Methods: PTX coated balloons (W.L. Gore, Flagstaff Ariz.) (5×40 mm) were built and coated with seven different coating formulations containing with different levels of stearic acid, and tris according to FIG. 8. The PTX loading on coated balloons was constant on all groups (3.5 μg/mm²). Devices with formulation 100-100-100 represented the control group and had PTX-stearic-tris levels as described in Publication No. 2017/0367705 (PTX: 3.5 μg/mm², Stearic: 0.12 μg/mm², Tris: 0.05 μg/mm²). Devices with formulation 100:0:0 had PTX only and included no excipients. All devices were sterilized and then deployed in both in vitro and in vivo environments.

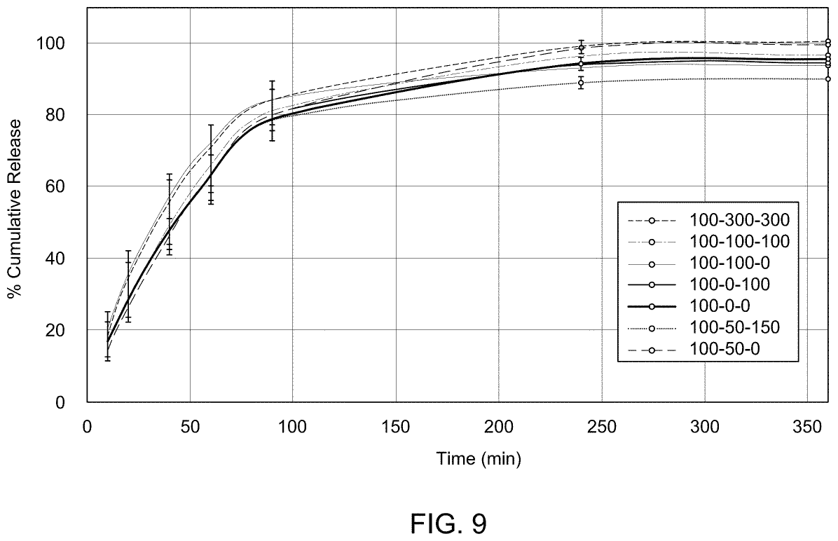

Results: As shown in FIG. 9, all devices exhibited similar PTX in vitro release profiles, regardless of the excipient level.

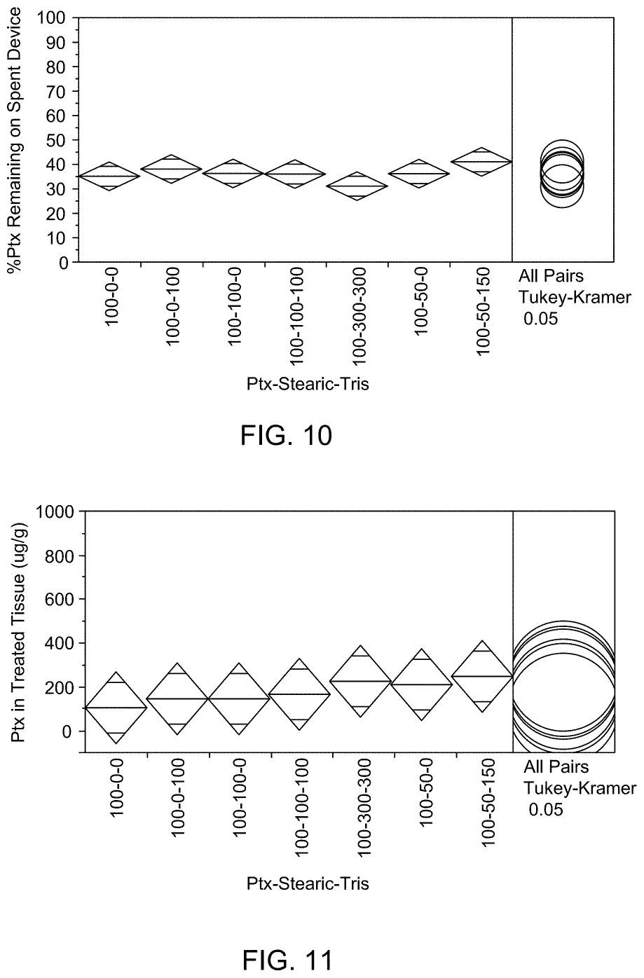

As shown in FIG. 10, excipient levels had no effect on the amount of PTX released from the balloons during in vivo deployment. All devices had approximately 35% PTX remaining on the balloon surface after in vivo deployment. FIG. 11 shows that excipient loading had no effect on PTX tissue levels 1 day after device deployment. Arteries treated with PTX-only coated balloons (100-0-0) resulted in 1d PTX tissue levels of approximately 100 μg PTX/g tissue PTX tissue levels were not significantly different (p>0.05) among the coating formulations.

Conclusions: These results suggest that excipient levels may have minimal effect, if any, on in vitro PTX release from an ePTFE balloon surface, as it appears that the PTX-only coating without excipients delivers similar drug levels as the control coating with excipients. Further, there are no significant differences across treatment means for the percentage of PTX remaining on devices after deployment and the retention of PTX in the treated tissue at one day.

### Example C

The preclinical performance of an embodiment of a DCB was evaluated, where PTX was applied to an ePTFE porous surface of a composite ePTFE/Nylon balloon. One of the testing groups included a drug coating containing stearic acid and tris excipients (formulation 100-100-100 in FIG. 8), while the other testing group included a drug-only coating (formulation 100-0-0 in FIG. 8, with no excipients).

Methods: PTX coated balloons (W.L. Gore, Flagstaff Ariz.) with dose densities of 3.5 μg/mm² (5×40 mm and 6×40 mm) were built, coated and sterilized. The devices were then deployed in the peripheral arteries of Yorkshire swine, and the PTX tissue concentrations were measured at one day, three days, seven days, and twenty-eight days after treatment.

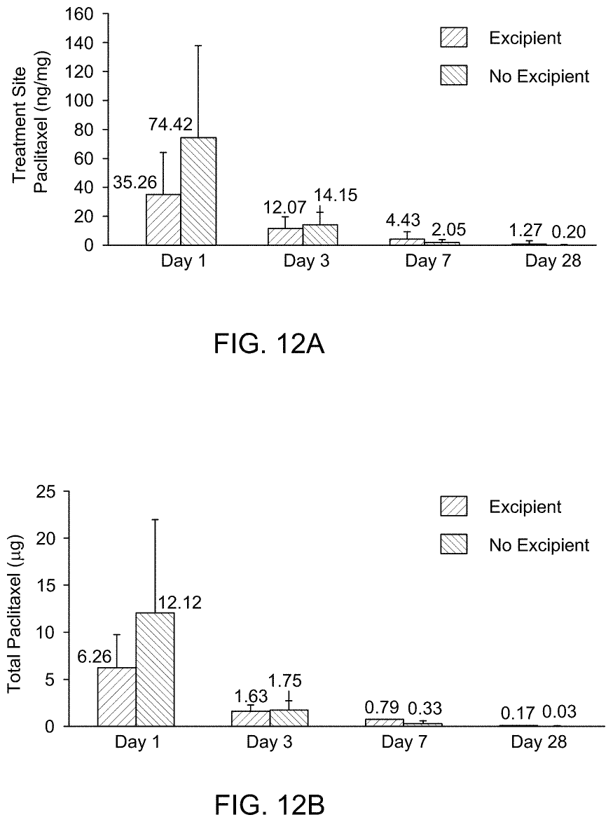

Results: As shown in FIG. 12A, the average arterial tissue concentration of PTX at the treatment site using a drug-only coated balloon was approximately 74.42 ng/mg at one day, 14.15 ng/mg at three days, 2.05 ng/mg at seven days, and 0.20 ng/mg at twenty-eight days. The average arterial tissue concentration of PTX at the treatment site using a balloon including excipients was approximately 35.26 ng/mg at one day, 12.07 ng/mg at three days, 4.43 ng/mg at seven days, and 1.27 ng/mg at twenty-eight days. As shown in FIG. 12B, the average total amount of PTX within the artery treated using a drug-only coated balloon was 12.12 μg at one day, 1.75 μg at three days, 0.33 μg at seven days, and 0.03 μg at twenty-eight days. The average total amount of PTX within the artery treated using a balloon including excipients was approximately 6.26 μg at one day, 1.63 μg at three days, 0.79 μg at seven days, and 0.17 μg at twenty-eight days.

Conclusions: At one day and three days, PTX tissue levels trended higher with the no excipient formulation, but this trend was later reversed at seven days and twenty-eight days.

### Example D

The preclinical performance of an embodiment of a DCB was evaluated, where PTX was applied to an ePTFE porous surface of a composite ePTFE/Nylon balloon and to a non-porous surface of a Nylon balloon. One of the testing groups for each balloon type included a drug coating containing stearic acid and tris excipients, while the other testing group for each balloon type included a drug-only coating without excipients.

Methods: PTX coated composite ePTFE/Nylon balloon balloons (W.L. Gore, Flagstaff Ariz.) (5×40 mm) were built, coated, packaged, and sterilized. One group of PTX coated balloons included stearic acid and tris excipients (formulation 100-100-100 in FIG. 8), while another group of PTX coated balloons did not include excipients (formulation 100-0-0 in FIG. 8). Additionally, two groups of Nylon balloons were coated with PTX, packaged and sterilized, again one group with excipients (formulation 100-100-100 in FIG. 8) and the other group without excipients (formulation 100-0-0 in FIG. 8). The target PTX loading for each device was 2.6 mg. Each group underwent in vitro dissolution testing. The composite balloon groups additionally underwent introducer valve crossing, with the PTX remaining on the device and the PTX loss to the packaging sheath and introducer calculated as a percentage of the target loading.

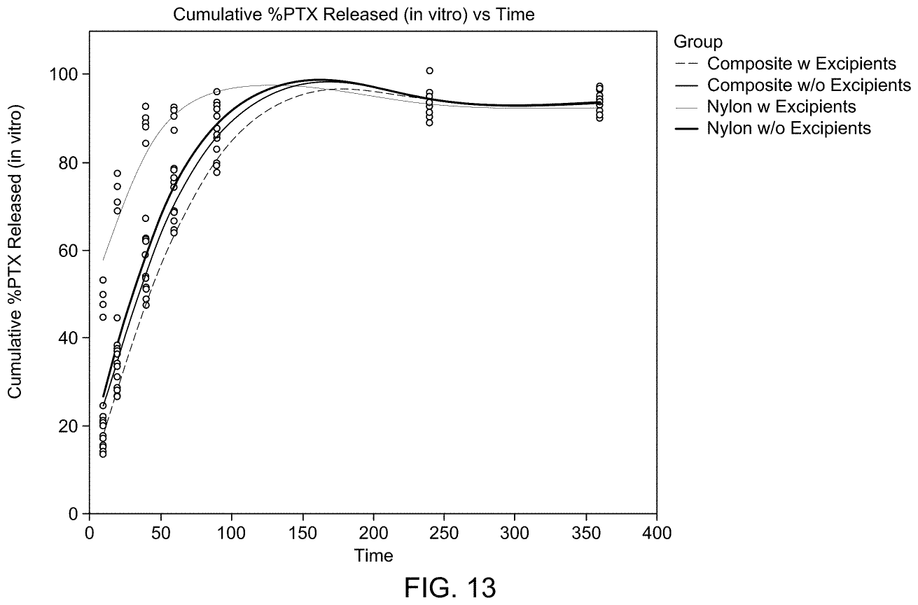

Results: As shown in FIG. 13, both the nylon balloon and composite balloon with PTX only (no excipients) had similar in vitro release as the composite balloon coated with PTX and excipients. These three device groups exhibited similar burst release and final cumulative release. The nylon balloon coated with PTX and excipients showed a higher burst release than the other 3 device groups.

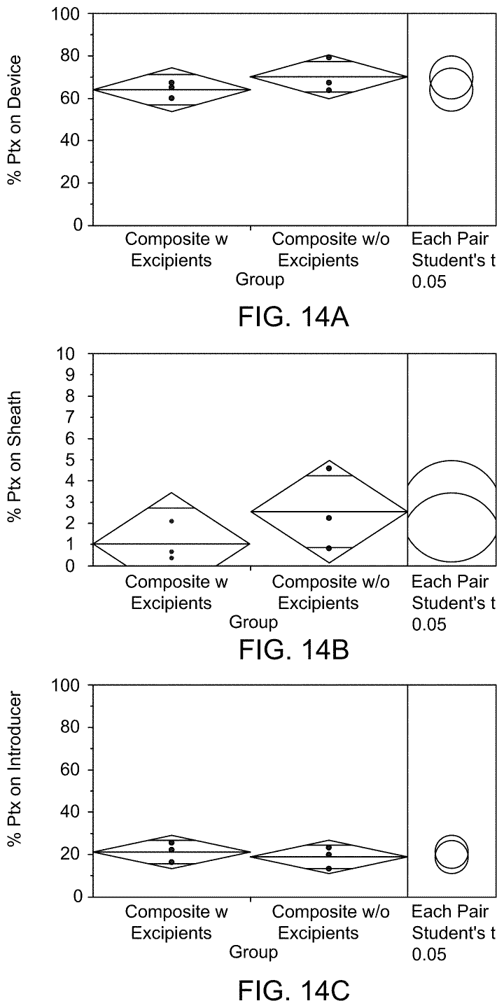

As shown in FIG. 14A, after introducer value crossing there was no significant difference (p>0.05) in the average amount of PTX remaining on composite balloons coated with or without excipients. Likewise, there was no significant difference between formulations for the PTX loss to the packaging sheath or introducer valve (see FIGS. 14B and 14C respectively).

Conclusions: The PTX dissolution profiles suggest that the excipients have little effect on in vitro PTX dissolution, as the differences in results were not found to be significant. Additionally, the PTX content on the composite balloons, packaging sheaths, and introducers was not significantly different across the tested groups, suggesting that a composite balloon without excipients can be passed through an introducer valve without increased drug loss when compared to a composite balloon coated with excipients.

### Example E

The in vivo performance of an embodiment of a DCB was evaluated where PTX was applied to an ePTFE porous surface of a composite ePTFE/Nylon balloon). DCBs with a drug-coating including excipients (formulation 100-100-100 in FIG. 8), were compared comprehensively with DCBs with a drug-coating layer excluding excipients (formulation 100-0-0 in FIG. 8).

Methods: PTX coated balloons with and without excipients (5 mm×40 mm and 6 mm×40 mm ePTFE— W.L. Gore, Flagstaff Ariz.) were inflated in peripheral arteries of a Familial Hypercholoesterolemic swine in-stent restenosis model. Two commercially available balloons—one being a PTX coated nylon balloon with excipients (Commercial 1, Medtronic IN.PACT™ Admiral™ Paclitaxel-coated PTA Balloon Catheter) and another being an uncoated nylon balloon (Commercial 2, Cordis POWERFLEX® PRO PTA Dilatation Catheter)—were also inflated in peripheral arteries of a Familial Hypercholoesterolemic swine in-stent restenosis model. Briefly, the model was created by injuring the treatment sites on Day 0. The injury was created by overstretching the artery with a standard angioplasty balloon catheter at a target 130% overstretch for three inflations at the site, followed by deployment of a bare metal self-expandable stent. Stents were selected to target approximately a 20% overstretch. On approximately Day 15, the injured, stented sites were imaged with angiography and optical coherence tomography (OCT) followed by treatment with the balloons. Injured sites received a single treatment from each balloon and were inflated for 60 seconds. Following treatment, angiography was repeated. On approximately Day 45, the injured, treated sites underwent repeat imaging with angiography and OCT. Images collected at Days 0, 15, and 45 were used for quantitative vascular angiography (QVA) analysis. The treated peripheral arteries, along with other tissues, were collected and prepared for light microscopy analysis and histomorphometry.

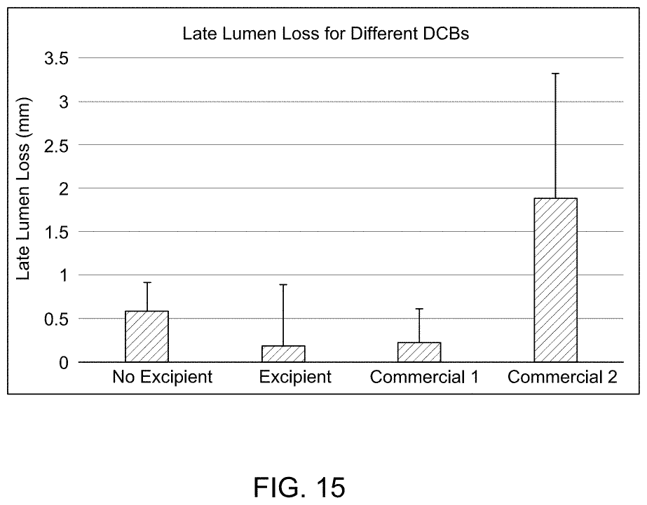

Results: As shown in FIG. 15, arteries treated with composite balloons including excipients experienced, on average, approximately 0.18 mm of late lumen loss, while arteries treated with composite balloons excluding excipients experienced, on average, about 0.58 mm of late lumen loss. Arteries treated with the Commercial 1 coated balloons experienced, on average, approximately 0.22 mm of late lumen loss, and arteries treated with the Commercial 2 uncoated balloons experienced, on average, approximately 1.88 mm of late lumen loss.

Conclusions: The results suggest that excipients have little effect on late lumen loss, as the differences between the drug-coated balloons tested were not significant.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A drug coated balloon, comprising:
a balloon having an outer surface comprised of expanded polytetrafluoroethylene; and
a drug coating layer on the outer surface of the balloon, the drug coating layer comprising at least one therapeutic agent and being substantially free of excipients.

2. The drug coated balloon of claim 1, the balloon being comprised of a composite of expanded polytetrafluoroethylene and nylon.

3. The drug coated balloon of claim 1, wherein the at least one therapeutic agent comprises paclitaxel.

4. The drug coated balloon of claim 3, wherein the balloon is configured to deliver the paclitaxel to a tissue to reduce a cellular proliferative response associated with restenosis.

5. The drug coated balloon of claim 3, wherein the drug coating layer further comprises a therapeutic agent selected from docetaxel, protaxel, arsenic trioxide, thalidomide, atorvastatin, cerivastatin, Fluvastatin, betamethasone diproprionate, dexamethasone 21-palmitate, sirolimus, everolimus, zotarolimus, biolimus, or temsirolimus.

6. The drug coated balloon of claim 1, wherein the drug coating layer contains from 0% to 4.75% by weight of the excipients.

7. The drug coated balloon of claim 1, wherein the drug coating layer contains from 0% to 4.75% by weight of the excipients, wherein the excipients are selected from fatty acids and their derivatives and urea.

8. The drug coated balloon of claim 1, wherein the drug coating layer comprises microcrystals in a haystack orientation having a random and a substantial absence of uniformity in placement on the outer surface of the balloon.

9. The drug coated balloon of claim 1, wherein the drug coating layer penetrates the outer surface of the balloon by an average penetration depth of 2 μm to 10 μm.

10. The drug coated balloon of claim 1, wherein about 80% of the drug coating layer releases from the balloon in about 100 minutes following implantation.

11. A drug coated balloon comprising:
a balloon having an outer surface; and
a drug coating layer on the outer surface of the balloon, the drug coating layer comprising at least one therapeutic agent and being substantially free of excipients;
wherein the drug coating layer comprises microcrystals in a haystack orientation having a random and a substantial absence of uniformity in placement on the outer surface of the balloon.

12. The drug coated balloon of claim 1, the outer surface further comprising nylon.

13. The drug coated balloon of claim 8, wherein a majority of the microcrystals each have a major dimension length that is at least 10 times greater than a major dimension width.

14. The drug coated balloon of claim 13, wherein the major dimension length of the majority of the microcrystals is at least 13 times or at least 15 times greater than the major dimension length.

15. The drug coated balloon of claim 13, wherein the major dimension width of the majority of the microcrystals is between 0.5 μm and 2 μm.

16. The drug coated balloon of claim 8, wherein the microcrystals have a random and substantial absence of uniformity in angles from the outer surface, and a majority of the microcrystals project from the outer surface at an angle of 50 degrees to 15 degrees.

17. The drug coated balloon of claim 1, the outer surface comprised of expanded polytetrafluoroethylene.

18. A method for preparing a vessel for drug application, the method comprising the steps of:
solubilizing at least one therapeutic agent in a solvent to produce a solution, the solution being substantially free of excipients;
coating an outer surface of a medical balloon with the solution; and
evaporating the solvent, leaving a drug coating layer comprising the at least one therapeutic agent on the outer surface of the balloon so that the drug coating layer comprises microcrystals in a haystack orientation having a random and substantial absence of uniformity in placement on the outer surface of the balloon.

19. The method of claim 18, wherein the solvent comprises acetone.

20. The method of claim 19, wherein the solvent further comprises water.

21. The method of claim 20, wherein the solvent comprises approximately 75% acetone and approximately 25% water.

22. The method of claim 20, wherein the solvent further comprises dioxane.

23. The method of claim 22, wherein the solvent comprises approximately 58% acetone, 14% dioxane, and 28% water.

24. The method of claim 18, wherein the balloon is comprised of a composite material comprising a fluoropolymer and nylon.

25. The method of claim 18, wherein the at least one therapeutic agent comprises paclitaxel.

26. A drug coated balloon, comprising:
a balloon having an outer surface comprised of expanded polytetrafluoroethylene; and
a drug coating layer on the outer surface of the balloon, the drug coating layer comprising at least one therapeutic agent and from 0% to 4.75% by weight of fatty acids and their derivatives.

27. The balloon of claim 26, wherein the fatty acids and their derivatives are selected from monocarboxylic acids, polysorbates, and shellac.

28. A drug coated balloon, comprising:
a balloon having an outer surface comprised of expanded polytetrafluoroethylene; and
a drug coating layer on the outer surface of the balloon, the drug coating layer comprising at least one therapeutic agent and from 0% to 4.75% by weight of urea.
